(19) United States
(12) Patent Application Publication
(10) Pub. No.: US 2022/0304952 A1
(43) Pub. Date: Sep. 29, 2022

(54) COMPOSITION COMPRISING METFORMIN HCI, VITAMIN B12 AND AT LEAST ONE FLOW ADDITIVE

(71) Applicant: DSM IP ASSETS B.V., Heerien (NL)

(72) Inventors: Martin Thomas KUENTZ, Kaiseraugst (CH); Zdravka MISIC, Kaiseraugst (CH)

(21) Appl. No.: 17/619,474

(22) PCT Filed: Jun. 17, 2020
(86) PCT No.: PCT/EP2020/066768

§ 371 Date: Dec. 15, 2021

(30) Foreign Application Priority Data
Jun. 17, 2019 (EP) 19180449.1

(51) Int. Cl. A61K 31/155; A61K 31/714; A61K 9/20

(52) U.S. Cl. CPC A61K 31/155; A61K 31/714; A61K 9/2009

(57) ABSTRACT

The present invention relates to a composition comprising a) metformin HCl, b) vitamin B12 and c) at least one flow additive. The flow additive comprises a calcium salt. The preferred calcium salt is calcium phosphate. The composition has good flowability and can be used to manufacture a solid pharmaceutical dosage form.

### TECHNICAL FIELD

The present invention relates to the manufacturing of solid oral dosage forms comprising metformin HCl.

### BACKGROUND OF THE INVENTION

Metformin HCl is a known active pharmaceutical ingredient (API). Solid dosage forms comprising metformin HCl are commercially available.

Metformin HCl is a very poorly flowable powder.

When manufacturing pharmaceutical dosage forms, flowability is important. Poor powder flow results in poor quality products, lower production rate, rejected batches and possibly product recalls. Flowability of powders can be improved by (i) granulation or (ii) by adding flow additives. WO 2016/096997 discloses the use of surface-reacted calcium carbonate for improving the flowability of a pharmaceutical delivery system. EP 2 938 362 A1 discloses a dry granulation process for producing tablet compositions of metformin and compositions thereof.

During metformin treatment, it is important to maintain the patient's vitamin B12 serum level.

CN101716182A discloses a combined medicine containing metformin hydrochloride and vitamin B12. In a preferred embodiment of CN101716182A, metformin hydrochloride and vitamin B12 are separately formed formulations packaged as a kit. However, pharmaceutical kits have many disadvantages:

patient compliance is poor, additional space and staff is required for kit packing, and monitoring of expiry dates is more complicated. In addition, kits trigger higher costs, such as additional handling and packing cost. These disadvantages can be overcome by a providing a fixed dose combination (FDC) instead of a kit. A fixed dose combination (FDC) may be a tablet, a capsule or a powder. Irrespective of the chosen form, a flowable powder is required. Said flowable powder may then be compressed into a tablet, may be filled into empty capsule shells or may be filled into sachets or stick-packs.

There is a need for a solid pharmaceutical composition that comprises both, metformin HCl and vitamin B12. To be suitable for manufacturing a fixed dose combination (FDC), the blend must be flowable and must have high content uniformity of metformin HCl and vitamin B12. The composition must be storage stable and is preferably directly compressible (DC). Excipients contained in the composition must be acceptable to major health authorities such as the US Food & Drug Administration (FDA).

### SUMMARY OF THE INVENTION

The problems underlying the present invention are solved by a composition comprising:
a) metformin HCl
b) vitamin B12, and
c) at least one flow additive,

wherein said at least one flow additive comprises or consists of at least one calcium salt.

The flow additive of the invention is a powder that essentially consists of particles. Said particles are preferably relatively large. Particularly good flowability is achieved when less than 10 wt.-% of the composition's flow additives are retained by mesh 40 and more than 30 wt.-% of the composition's flow additives are retained by mesh 100, based on the total weight of the flow additive. In a preferred embodiment, the flow additive of the invention comprises or consists of aggregates of primary particles. Thus, the present invention also relates to the use of aggregates of primary particles for increasing the flowability of a composition that comprises metformin HCl and vitamin B12, wherein said aggregates comprise or consist of at least one calcium salt.

Any kind of calcium salt can be used as long as the chosen calcium salt maintains or increases the flowability of the composition of the invention. The preferred calcium salt of the invention is anhydrous dicalcium phosphate (CaHPO₄).

If the composition of the invention comprises a relatively large amount of flow additive, particularly high flowability is achieved. In a preferred embodiment of the invention, the weight ratio between metformin HCl and the at least one flow additive is from 200:1 to 1:1, more preferably from 100:1 to 1:1, even more preferably from 50:1 to 1:1 and most preferably from 10:1 to 1:1.

The composition of the invention comprises preferably a spray dried formulation of vitamin B12. If the composition of the invention comprises a spray dried formulation of vitamin B12 (instead of vitamin B12 crystals), content uniformity of vitamin B12 in the obtained fixed-dose combination is particularly good. In addition, storage stability of the obtained fixed-dose combination is good or very good.

The present invention also relates to a method of manufacturing a fixed-dose combination of metformin HCl and vitamin B12, said method comprising the step of filling the herein described composition into a container (e.g. a sachet or a capsule shell) or into a die (e.g. into a die of a tablet press). In a preferred embodiment of the method of the invention, the herein described composition flows down a guiding tool into a container or into a die.

The fixed-dose combination of the invention is a solid oral dosage form such as a tablet, a capsule, a sachet or a stick-pack. Patients that are being treated with the solid oral dosage form of the invention maintain or regain a healthy vitamin B12 serum level. Thus, the present invention also relates to herein described solid oral dosage form for use in the treatment or prevention of metformin induced vitamin B12 depletion.

In comparison with a kit, the fixed-dose combination of the invention has improved patient compliance. Thus, the present invention also relates to a method for increasing patient compliance, wherein the herein described solid oral dosage form is administered to patients that are in need of metformin and vitamin B12 supplementation.

### DETAILED DESCRIPTION OF THE INVENTION

The present invention relates to a composition that comprises (a) metformin HCl, (a) vitamin B12, and (b) at least one flow additive. The composition of the invention comprises preferably a spray dried formulation vitamin B12. Vitamin B12 is preferably cyanocobalamin. In the context of the present invention, vitamin B12 is considered to be an active pharmaceutical ingredient.

### Definitions

The abbreviation “wt.-%” means weight-%.

The term “comprising” is an open term. Therefore, the herein described composition may comprise more than one kind of flow additive. Similarly, the herein described composition may comprise more than one kind of spray dried vitamin B12 formulation. However, preferably, the herein described composition comprises one kind of spray dried vitamin B12 formulation only. Typically, the composition of the invention comprises several pharmaceutically acceptable excipients, wherein one of the pharmaceutically acceptable excipients is a flow additive. Thus, an illustrative embodiment of the invention relates to a composition comprising:
a) metformin HCl
b) one spray dried formulation of vitamin B12,
c) one flow additive, and
d) at least one further pharmaceutically acceptable excipient,

wherein said at least one flow additive comprises or consists of at least one calcium salt.

The solid pharmaceutical dosage form of the invention comprises the herein described composition. In the context of the present invention, the term “solid pharmaceutical dosage form” refers to a dosage form such as a tablet, a capsule and a powder. Powders (such as powders for oral solution) are typically packaged in a sachet or a stick-pack. Alternatively, powders may be filled into two-piece capsules (e.g. gelatine capsules size 0, 00 or 000). In a preferred embodiment of the invention, the term “solid pharmaceutical dosage form” refers to a solid oral pharmaceutical dosage form selected from the group consisting of tablets, capsules and powders. In an even more preferred embodiment of the invention, the term “solid pharmaceutical dosage form” refers to a compressed tablet.

The solid pharmaceutical dosage form of the invention comprises preferably microcrystalline cellulose (MCC). MCC is a well-known excipient prepared by acid hydrolysis of cellulose. On industrial scale, MCC is obtained by hydrolysis of wood and/or cotton cellulose using dilute mineral acids. The treated pulp is then rinsed and spray-dried with or without an additional process step such as milling. Numerous types of microcrystalline cellulose (MCC) are available on the market. In the context of the present invention, the term “microcrystalline cellulose” includes any type of microcrystalline cellulose consisting of partially depolymerized cellulose such as the excipients listed in Table 1 of T. Vehovec et al.: “Influence of different types of commercially available microcrystalline cellulose on degradation of perindopril erbumine and enalapril maleate in binary mixtures”, Acta Pharm. 62 (2012), page 518. Also included is silicified microcrystalline cellulose such as PROSOLV® SMCC. In the context of the present invention, the term “silicified microcrystalline cellulose” refers to an excipient comprising microcrystalline cellulose (MCC) and silicon dioxide such as colloidal silicon dioxide (CSD).

Vitamin B12 is a well-known water-soluble vitamin. In the context of the present invention, the term “vitamin B12” refers to any vitamer of vitamin B12 and includes vitamin B12 derivatives and/or metabolites of vitamin B12. Preferably, however, the term “vitamin B12” refers to cyanocobalamin. Cyanocobalamin may be produced by fermentation using suitable microorganisms.

“Crystalline vitamin B12” comprises at least 98 weight-% vitamin B12, based on the total weight of the crystals. Preferably, the composition of the invention does not comprise any crystalline vitamin B12.

The composition of the invention comprises preferably at least one spray dried formulation of vitamin B12. The expression “spray dried formulation of vitamin B12” refers to a powder which is obtainable by spray drying of an aqueous solution that comprises vitamin B12 and at least one excipient, wherein said at least one excipient is preferably selected from the group consisting of sodium citrate, trisodium citrate, citric acid, maltodextrin citric acid and modified food starch. In a preferred embodiment of the invention, the expression “spray dried formulation of vitamin B12” refers to a powder which is obtainable by spray drying an aqueous solution which comprises cyanocobalamin and at least one excipient, wherein said at least one excipient is preferably selected from the group consisting of sodium citrate, trisodium citrate, citric acid, maltodextrin and modified food starch.

Vitamin B12 crystals have a vitamin B12 content of at least 98 weight-%, based on the total weight of the crystals. Due to the presence of at least one excipient, the spray dried formulation of vitamin B12 comprises less than 90 weight-% of vitamin B12, based on the total weight of the spray dried formulation. The exact concentration of vitamin B12 in the spray dried formulation of vitamin B12 depends on the amount of excipient in the spray dried formulation. Preferably, the spray dried formulation of vitamin B12 of the invention comprises 1 weight-% or less of vitamin B12, based on the total weight of the spray dried formulation. The person skilled in the art understands that spray dried formulations of vitamin B12 being free of vitamin B12 are excluded. Also preferably, the spray dried formulation of vitamin B12 of the invention is a water-soluble or water-dispersible powder comprising 1 weight-% or less of cyanocobalamin, based on the total weight of the powder. The person skilled in the art understands that powders being free of vitamin B12 are excluded. In the most preferred embodiment of the invention, the expression “spray dried formulation of vitamin B12” refers to a powder which is obtainable by spray drying an aqueous solution which comprises cyanocobalamin and at least one excipient, wherein said excipient is preferably selected from the group consisting of sodium citrate, trisodium citrate, citric acid, maltodextrin and modified food starch, and wherein said powder comprises 1 weight-% or less of cyanocobalamin, based on the total weight of the powder. Again, the person skilled in the art understands that powders being free of vitamin B12 are excluded.

In the context of the present invention, the term “metformin” refers to metformin or to a pharmaceutically acceptable salt thereof. The probably best known pharmaceutically acceptable salt of metformin is metformin HCl. Therefore, in the most preferred embodiment of the invention, the term “metformin” refers to metformin HCl.

Metformin HCl has a poor compactability and flowability. Therefore, metformin HCl is preferably granulated before tableting. During such granulation process, metformin is transformed into free-flowing, essentially dust-free granules that are easy to compress. In the context of the present invention, the term “granulated metformin” refers to granules comprising the herein described composition. Thus, the term “granulated metformin” refers to granules comprising metformin HCl, at least one spray dried formulation of vitamin B12 at least one flow additive and preferably at least one further pharmaceutically acceptable excipient. Typically, said at least one further excipient is a binder and/or a lubricant. Suitable binders are listed for example in Arndt et al., “Roll Compaction and Tableting of High Loaded Metformin Formulations Using Efficient Binders”, AAPS PharmSciTech, July 2018, Volume 19, Issue 5, pp 2068-2076.

In the context of the present invention, the term “calcium salt” refers to any pharmaceutically acceptable calcium salt. Thus, the term includes calcium phosphate, calcium carbonate and calcium citrate. Calcium carbonate is a chemical compound with the formula CaCO₃. The term “calcium citrate” includes monocalcium citrate, dicalcium citrate and tricalcium citrate. Known tricalcium citrate salts include anhydrous calcium citrate (i.e. Ca₃(C₆H₅O₇)₂) and tricalcium dicitrate tetrahydrate (i.e. [Ca₃(C₆H₅O₇)₂(H₂O)₂].2H₂O). The term “calcium phosphate” includes anhydrous calcium phosphate and hydrous calcium phosphate. Known are anhydrous calcium phosphates, anhydrous monocalcium phosphate (Ca(H₂PO₄)₂), anhydrous dicalcium phosphate (CaHPO₄) or anhydrous tricalcium phosphate (Ca₃(PO₄)₂). In the most preferred embodiment of the invention, the term calcium salt refers to anhydrous dicalcium phosphate (CaHPO₄).

Three major types of flow aids are known: mechanical flow aids, pneumatic flow aids and flow additives. The “flow additive” of the present invention is a solid composition that, when mixed with a poorly flowable product, improves flowability of the poorly flowable product. In the context of the present invention, the poorly followable product is a mixture comprising metformin HCl and vitamin B12 such as a spray dried formulation of vitamin B12. Typically, the flow additive of the present invention is a powder. Any powder comprises or consists of particles. In a preferred embodiment of the invention, the flow additive comprises or consists of secondary particles, wherein each secondary particle is an aggregate of primary particles.

“Uniformity of content” ensures that a consistent dose of an active pharmaceutical ingredient (e.g. of vitamin B12) is maintained in each portion of a composition, even if the composition is subdivided into a large number of very small portions. By way of example, when controlling the quality of e.g. capsules or tablets, uniformity of content is determined. To do so, multiple e.g. capsules or tablets are selected at random and a suitable analytical method is applied to assay the individual content of the active pharmaceutical ingredient in each capsule or tablet. The relative standard deviation (RSD) can then be calculated. The lower the RSD, the better the uniformity of content. An RSD of more than 80% is clearly unacceptable. The term “uniformity of vitamin B12 content” is used when multiple solid pharmaceutical dosage forms are selected at random and a suitable analytical method is applied to assay the individual content of vitamin B12 in each solid pharmaceutical dosage form.

### Composition of the Invention

The composition of the present invention is preferably a mixture that comprises metformin HCl, at least one spray dried formulation of vitamin B12, and at least one flow additive. The flow additive is preferably a powder that comprises or consists of at least one calcium salt.

A powder comprises or consists of solid particles. Some commercially available powders comprise aggregates of primary particles. Such aggregates may be referred to as secondary particles. An example of such a powder is the filler Di-Cafos® (available at Budenheim). Di-Cafos® is a powder that comprises aggregates of primary particles, wherein said primary particles comprise or consist of dibasic calcium phosphate such as anhydrous dicalcium phosphate (CaHPO₄) or dihydrate dicalcium phosphate (CaHPO₄.2H₂O). An also known filler is Tri-Cafos®. Tri-Cafos® is a powder that comprises aggregates of primary particles, wherein said primary particles comprise or consist of tribasic calcium phosphate such as anhydrous tricalcium phosphate (Ca₃(PO₄)₂). According to the invention, dibasic calcium phosphate or tribasic calcium phosphate can be used. Preferred, however, is dibasic calcium phosphate, wherein anhydrous dicalcium phosphate (CaHPO₄) is particularly preferred.

Thus, one embodiment of the present invention relates to a composition comprising:
a) metformin HCl
b) at least one spray dried formulation of vitamin B12, and
c) at least one flow additive,

wherein said at least one flow additive is a powder which comprises aggregates of primary particles, and wherein said primary particles comprise or consist of dibasic calcium phosphate, and wherein said dibasic calcium phosphate is preferably anhydrous dicalcium phosphate (CaHPO₄), or

wherein said at least one flow additive is a powder which comprises aggregates of primary particles, and wherein said primary particles comprise or consist of tribasic calcium phosphate.

In one embodiment, the composition of the invention comprises at least one flow additive wherein
less than 10 wt.-%, preferably less than 6 wt.-% of the composition's flow additives pass mesh 325, based on the total weight of the flow additive, and/or
less than 30 wt.-%, preferably less than 26 wt.-% of the composition's flow additives pass mesh 200, based on the total weight of the flow additive, and/or
more than 30 wt.-%, preferably more than 35 wt.-% of the composition's flow additives are retained by mesh 100, based on the total weight of the flow additive, and/or
less than 10 wt.-%, preferably less than 5 wt.-% of the composition's flow additives are retained by mesh 40, based on the total weight of the flow additive.

A particle size conversion table is given below:

| | |
|---|---|
| Sieve Designation | Nominal Sieve Opening |

| Standard | Mesh | inches | mm | Microns |
|---|---|---|---|---|
| 0.500 mm | No. 35 | 0.0197 | 0.500 | 500 |
| 0.420 mm | No. 40 | 0.0165 | 0.420 | 420 |
| 0.354 mm | No. 45 | 0.0139 | 0.354 | 354 |
| 0.297 mm | No. 50 | 0.0117 | 0.297 | 297 |
| 0.250 mm | No. 60 | 0.0098 | 0.250 | 250 |
| 0.210 mm | No. 70 | 0.0083 | 0.210 | 210 |
| 0.177 mm | No. 80 | 0.0070 | 0.177 | 177 |
| 0.149 mm | No. 100 | 0.0059 | 0.149 | 149 |
| 0.125 mm | No. 120 | 0.0049 | 0.125 | 125 |
| 0.105 mm | No. 140 | 0.0041 | 0.105 | 105 |
| 0.088 mm | No. 170 | 0.0035 | 0.088 | 88 |
| 0.074 mm | No. 200 | 0.0029 | 0.074 | 74 |
| 0.063 mm | No. 230 | 0.0025 | 0.063 | 63 |
| 0.053 mm | No. 270 | 0.0021 | 0.053 | 53 |
| 0.044 mm | No. 325 | 0.0017 | 0.044 | 44 |

Thus, a preferred embodiment of the present invention relates to a composition comprising:
a) metformin HCl
b) at least one spray dried formulation of vitamin B12, and
c) at least one flow additive,

wherein said at least one flow additive comprises aggregates of primary particles, wherein said primary particles comprise or consist of anhydrous dicalcium phosphate (CaHPO₄), and/or

wherein less than 10 wt.-% of the flow additive is retained by mesh 40 and more than 30 wt.-% of the flow additive is retained by mesh 100, based on the total weight of the flow additive.

In most cases, flow additives impart functionality at relatively low concentrations. While many people assume that more flow additive will equate to better flow properties, the person skilled in the art knows that this assumption is most often wrong (“Powder Handling: Make the Most of Flow Additives” by: Armstrong, B.; Clayton, J. Chemical Processing, 2014, 77(4)). Despite of this general knowledge, the composition of the invention comprises preferably a relatively large amount of flow additive. Preferably, the weight ratio between metformin HCl and the at least one flow additive is from 200:1 to 1:1 and is more preferably from 100:1 to 1:1 and is even more preferably from 50:1 to 1:1 and is most preferably from 30:1 to 1:1. Thus, a preferred embodiment of the present invention relates to a composition comprising:
a) metformin HCl
b) at least one spray dried formulation of vitamin B12, and
c) at least one flow additive,

wherein said at least one flow additive comprises aggregates of primary particles, wherein said primary particles comprise or consist of anhydrous dicalcium phosphate (CaHPO₄), and

the weight ratio between metformin HCl and the at least one flow additive is from 200:1 to 1:1 and is more preferably from 100:1 to 1:1 and is even more preferably from 50:1 to 1:1 and is most preferably from 30:1 to 1:1.

Ensuring uniformity of vitamin B12 content in each portion of a composition is extremely difficult. When using commercially available vitamin B12 crystals, uniformity of vitamin B12 content is often very poor. In some trials, relative standard deviations (RSD) values of more than 90% were measured.

Uniformity of vitamin B12 content can be tremendously improved when using a spray dried formulation of vitamin B12 instead of using commercially available vitamin B12 crystals. The lower the concentration of vitamin B12 in the spray dried formulation, the higher the uniformity of vitamin B12 content. When using a spray dried formulation of vitamin B12 which contains 1 weight-% cyanocobalamin, based on the total weight of the spray dried formulation of vitamin B12, a relative standard deviation (RSD) of less than 4% can be achieved. When using in the same process a more diluted spray dried formulation of vitamin B12 containing only 0.1 weight-% cyanocobalamin, based on the total weight of the spray dried formulation of vitamin B12, RSD could be further lowered to 2%. Suitable spray dried formulation of vitamin B12 are commercially available as “Vitamin B12 1% SD” or “Vitamin B12 0.1% WS” from DSM® Nutritional Products (Switzerland). “Vitamin B12 1% SD” comprises 1 weight-% cyanocobalamin, based on the total weight of the respective product, whereas “Vitamin B12 0.1% WS” comprises 0.1 weight-% cyanocobalamin, based on the total weight of the respective product. According to the present invention, “Vitamin B12 0.1% WS” is the preferred spray dried formulation of vitamin B12.

Thus, a preferred embodiment of the present invention relates to a composition comprising:
a) metformin HCl
b) at least one spray dried formulation of vitamin B12, and
c) at least one flow additive,

wherein said at least one flow additive comprises aggregates of primary particles, wherein said primary particles comprise or consist of anhydrous dicalcium phosphate (CaHPO₄), and

wherein the weight ratio between metformin HCl and the at least one flow additive is from 200:1 to 1:1 and is more preferably from 100:1 to 1:1 and is even more preferably from 50:1 to 1:1 and is most preferably from 30:1 to 1:1, and

wherein said spray dried formulation of vitamin B12 comprises preferably from 0.01 to 1 weight-%, more preferably from 0.05 to 0.5 weight-% and most preferably 0.1 weight-% cyanocobalamin, based on the total weight of the spray dried formulation of vitamin B12.

The person skilled in the art knows how to manufacture such spray dried formulations of vitamin B12. In one embodiment, the herein described spray dried formulation of vitamin B12 is produced as disclosed in example 1 of U.S. Pat. No. 5,397,576.

Thus, a preferred embodiment of the present invention relates to a composition comprising:
a) metformin HCl
b) at least one spray dried formulation of vitamin B12, and
c) at least one flow additive,

wherein said at least one flow additive comprises aggregates of primary particles, wherein said primary particles comprise or consist of anhydrous dicalcium phosphate (CaHPO₄), and

wherein the weight ratio between metformin HCl and the at least one flow additive is from 200:1 to 1:1 and is more preferably from 100:1 to 1:1 and is even more preferably from 50:1 to 1:1 and is most preferably from 30:1 to 1:1, and

wherein said spray dried formulation of vitamin B12 comprises preferably from 0.01 to 1 weight-%, more preferably from 0.05 to 0.5 weight-% and most preferably 0.1 weight-% cyanocobalamin, based on the total weight of the spray dried formulation of vitamin B12, and/or

wherein the spray dried formulation of vitamin B12 is obtainable by spray drying of an aqueous solution that comprises vitamin B12 and at least one auxiliary compound, and wherein said at least one auxiliary compound is preferably selected from the group consisting of sodium citrate, trisodium citrate, citric acid, maltodextrin citric acid and modified food starch.

The composition of the invention is preferably a mixture, more preferably a powderous mixture. It has good flowability. The herein described composition has a flow rate of preferably at least 250 g/min, more preferably at least 500 g/min and most preferably at least 1000 g/min when measuring the flow rate of the composition through a funnel with an orifice that has an inner diameter of 15 mm.

The Carr index is used as an indication of the flowability of a powder. In the context of the present invention, the terms “Carr's index”, “Carr index” and “compressibility index” mean the same. The compressibility index (and thus also the Carr index) is calculated as follows:

Compressibility Index=100×[(ρ_{tapped}−ρ_{bulk})/ρ_{tapped}]

where

ρ_{tapped} is the tapped density of the powder.

ρ_{bulk} is the freely settled bulk density of the powder.

Preferably, the herein described composition has a Carr index of preferably less than 30, more preferably of less than 28 and most preferably of less than 25. Thus, the most preferred embodiment of the present invention relates to a composition comprising:
a) metformin HCl
b) at least one spray dried formulation of vitamin B12, and
c) at least one flow additive,

wherein said at least one flow additive comprises aggregates of primary particles, wherein said primary particles comprise or consist of anhydrous dicalcium phosphate (CaHPO₄), and

wherein the weight ratio between metformin HCl and the at least one flow additive is from 200:1 to 1:1 and is more preferably from 100:1 to 1:1 and is even more preferably from 50:1 to 1:1 and is most preferably from 30:1 to 1:1, and

wherein said spray dried formulation of vitamin B12 comprises 0.1 weight-% cyanocobalamin, based on the total weight of the spray dried formulation of vitamin B12, and

wherein the composition is a mixture having a Carr index of preferably less than 30, more preferably of less than 28 and most preferably of less than 25, and/or

wherein the composition is a mixture having a flow rate of preferably at least 250 g/min, more preferably at least 500 g/min and most preferably at least 1000 g/min when measuring the flow rate of the composition (i.e. of the mixture) through a funnel with an orifice that has an inner diameter of 15 mm.

### Solid Pharmaceutical Dosage Form

Preferably, the composition of the present invention is used for manufacturing a solid pharmaceutical dosage form. The thus manufactured solid pharmaceutical dosage form comprises the composition of the present invention. Preferably, the solid pharmaceutical dosage form is a tablet, a capsule or a powder in a sachet or in a stick-pack.

The solid pharmaceutical dosage form of the invention comprises preferably from 1 μg to 10 μg cyanocobalamin, more preferably from 1 μg to 6 μg cyanocobalamin and most preferably from 1 μg to 4 μg cyanocobalamin. In case the solid pharmaceutical dosage form is a tablet, the tablet of the present invention comprises preferably from 1 μg to 10 μg cyanocobalamin per tablet, more preferably from 1 μg to 6 μg cyanocobalamin per tablet and most preferably from 1 μg to 4 μg cyanocobalamin per tablet. As for safety, Tolerable Upper Intake Levels (known as ULs) are set for vitamins and minerals when evidence is sufficient. In the case of vitamin B12, there is no UL, as there is no human data for adverse effects from high doses.

The solid pharmaceutical dosage form of the present invention comprises preferably at least one further pharmaceutically acceptable excipient. Typically, the solid pharmaceutical dosage form of the invention has a mass of less than 5 g, preferably of less than 4 g, more preferably of less than 3 g and most preferably of less than 2 g.

In a preferred embodiment of the invention, the solid pharmaceutical dosage form comprises vitamin B12 and 1000 mg metformin HCl, 500 mg metformin HCl or 850 mg metformin HCl, wherein the weight ratio between vitamin B12 and metformin HCl is from 1:10.000.000 to 1:1.000, preferably from 1:5.000.000 to 1:2.000 and most preferably from 1:1.000.000 to 1:4.000. In an also preferred embodiment of the invention, the solid pharmaceutical dosage form is a tablet or capsule which comprises 1000 mg metformin HCl or 500 mg metformin HCl and 1 μg to 10 μg cyanocobalamin. In the most preferable embodiment, the solid pharmaceutical dosage form is a tablet which comprises 1000 mg metformin HCl and 1 μg to 4 μg cyanocobalamin. Typically, two of these tablets are given per day to reach a daily dose of 2000 mg metformin HCl.

Thus, a preferred embodiment of the invention relates to a solid pharmaceutical dosage form comprising:
a) metformin HCl
b) at least one spray dried formulation of vitamin B12, and
c) at least one flow additive,

wherein said at least one flow additive comprises a calcium salt, and

wherein said vitamin B12 is cyanocobalamin, and wherein the weight ratio between cyanocobalamin and metformin HCl is from 1:10.000.000 to 1:1.000, preferably from 1:5.000.000 to 1:2.000 and most preferably from 1:1.000.000 to 1:4.000.

In a preferred embodiment of the invention, the solid pharmaceutical dosage form is a tablet. For compressing a tablet, a filler might be needed or is at least recommended. Preferred fillers are microcrystalline cellulose and silicified microcrystalline cellulose. Thus, a preferred embodiment of the invention relates to a tablet comprising:
a) metformin HCl
b) at least one spray dried formulation of vitamin B12,
c) at least one flow additive, and
d) at least one filler

wherein said flow additive is a powder, and wherein said powder preferably comprises aggregates of primary particles, wherein said primary particles comprise or consist of a calcium salt, and/or

wherein said at least one filler is preferably microcrystalline cellulose or silicified microcrystalline cellulose.

Compressing a tablet is easier when the mixture comprising metformin is granulated before being compressed into tablets. For granulation, a binder is used. Therefore, the present invention also relates to a composition comprising:
a) granulated metformin HCl
b) at least one spray dried formulation of vitamin B12, and
c) at least one flow additive,

wherein said at least one flow additive comprises a calcium salt, and

wherein said vitamin B12 is cyanocobalamin, and wherein the weight ratio between cyanocobalamin and metformin HCl is from 1:10.000.000 to 1:1.000, preferably from 1:5.000.000 to 1:2.000 and most preferably from 1:1.000.000 to 1:4.000, and/or

the weight ratio between metformin HCl and the at least one flow additive is from 200:1 to 1:1 and is more preferably from 100:1 to 1:1 and is even more preferably from 50:1 to 1:1 and is most preferably from 30:1 to 1:1.

Method of Manufacturing the Solid Pharmaceutical Dosage Form of the Present Invention

The solid pharmaceutical dosage form of the present invention comprises two pharmaceutically active ingredients: metformin HCl and vitamin B12. Thus, the solid pharmaceutical dosage form of the present invention is a fixed-dose combination of metformin HCl and vitamin B12.

When manufacturing such solid pharmaceutical dosage form, the herein described composition is filled into capsule shells or is filed into sachets or is compressed into tablets. In either case, during the manufacturing process, the herein described composition flows down a guiding tool into a container. Said container may be a sachet, an empty, not-yet closed stick-pack or a component of a pill maker (e.g. a die). Thereby, any suitable guiding tool might be used such as a tube, hose, a duct, a channel or a conduit.

Thus, the present invention also relates to a method of manufacturing a fixed-dose combination of metformin HCl and vitamin B12, said method comprising the step of letting the herein described composition flow down a guiding tool,

wherein said guiding tool is preferably a tube, hose, a duct, a channel or a conduit and/or

wherein the herein described composition preferably flows down into a container, said container being preferably an open sachet, an open stick-pack or a component of a pill maker.

Medical Use and Method of Treatment

The present invention also relates to the herein described a solid pharmaceutical dosage form for use as a medicament. The herein described a solid pharmaceutical dosage form comprises metformin. Thus, one embodiment of the invention relates to a solid pharmaceutical dosage form for use in the treatment of a patient who is in need of metformin, wherein said solid pharmaceutical dosage form comprises:
a) metformin HCl
b) at least one spray dried formulation of vitamin B12, and
c) at least one flow additive, and

wherein said at least one flow additive comprises or consists of at least one calcium salt.

A patient suffering from diabetes may be in need of metformin. Therefore, the present invention also relates to the herein described a solid pharmaceutical dosage form for use in the treatment of diabetes. Thus, one embodiment of the invention relates to a solid pharmaceutical dosage form for use in the treatment of diabetes, wherein said solid pharmaceutical dosage form comprises:
a) metformin HCl
b) at least one spray dried formulation of vitamin B12, and
c) at least one flow additive, and

wherein said at least one flow additive comprises or consists of at least one calcium salt.

Use of a Calcium Salt as a Flow Additive

Finally, the present invention relates to the use of aggregates of primary particles for increasing flowability of a composition that comprises metformin HCl and vitamin B12, wherein said primary particles comprise or consist of calcium salt. In a preferred embodiment, the present invention relates to the use of aggregates of primary particles for increasing flowability of a composition comprising metformin HCl and vitamin B12, wherein said primary particles comprise or consist of anhydrous dicalcium phosphate (CaHPO₄), and/or wherein less than 10 wt.-% of the aggregates are retained by mesh 40 and more than 30 wt.-% of the aggregates are retained by mesh 100, based on the total weight of the flow additive.

### FIGURES

FIG. 1 shows the flow rates measured in Examples 1 and 2. On the x-axis, the inner diameter of the respective orifice is indicated in mm. On the y-axis, the respective flow rate is indicated in g/minute.

As expected, the larger the inner diameter of the orifice, the higher the flow rate. This applies to all tested compositions having a flow rate >0 g/min.

For a given size of the orifice, CaCO₃ 95MD has higher flow rate (g/min) than DiCafos A150. At an inner diameter of 15 mm, for example, the flow rate of CaCO₃ 95MD (2143 g/min) is higher than the flow rate of DiCafos A150 (1636 g/min). However, when looking at the mixtures instead of looking at the respective calcium salt only, the opposite is the case. This is surprising. At an inner diameter of 15 mm, for example, the flow rate of a mixture comprising metformin HCl, a spray dried formulation of vitamin B12 and CaCO₃ 95MD (163 g/min) is lower than the flow rate of a mixture comprising metformin HCl, a spray dried formulation of vitamin B12 and DiCafos A150 (909 g/min). At inner diameters of less than 15 mm (i.e. 10 mm, 9 mm, 7 mm and 5 mm), the flow rate of a mixture comprising metformin HCl, a spray dried formulation of vitamin B12 and CaCO₃ 95MD was even 0 g/min (i.e. the mixture did not flow at all).

DiCafos A150 is a commercially available powder comprising relatively large aggregates of primary particles.

In order to test the influence of the different sources of ionic calcium, four similar tablets were prepared. FIG. 2 shows compression profiles of the four tablets prepared in Example 3.

### EXAMPLES

### Example 1 (pretests)

In example 1, several pretests were done.

Flowability of the commercially available metformin HCl was measured as follows:

The powder flowability was determined with a Pharmatest PTG-S4 automated powder characterization instrument (Pharma Test Apparatebau AG, Hainburg, Germany). This system measures the flow behavior of granules and powders in compliance with the current EP <2.9.36/17> and USP <1174> pharmacopoeia as well as with the international ISO 4324 standards.

Mass flow rate (g/min) was determined via the method of flow through an orifice. Flow rate is interpreted as the time needed for a specified amount of powder (100 g) to flow through an orifice with different diameters. A free-flowing powder should be able to flow through the whole set of diameters 5, 7, 9, 10, and 15 mm. The plot of flow rate vs. orifice diameter is referred as flow curve. Three parallel measurements were performed to determine the flow rate.

As expected, the flow rate of metformin HCl as such was very low even when using the largest orifice (i.e. inner diameter=15 mm).

Then, flowability of (a) DiCafos A150 (dicalciumphosphat anhydrous, available at Budenheim) and (b) CaCO₃ 95MD (calcium carbonate, available at Particle Dynamics) was measured in the same manner, using several sizes of orifices.

Independent of the size of the orifice, CaCO₃ 95MD had higher flow rate (g/min) than DiCafos A150.

The results of these flowability tests suggest the following:
1. For manufacturing of a solid oral dosage form, flowability of metformin HCl needs to be improved.
2. Both, DiCafos A150 and CaCO₃ 95MD could be used as pharmaceutically acceptable flow additive.
3. CaCO₃ 95MD is the better flow additive than DiCafos A150.

As a further pretest, Carr index was measured:

Carr index of CaCO₃ 95MD was lower (15.8) than Carr index of DiCafos A150 (17.4). As a rule of thumb, the lower the Carr index, the better the flowability. Therefore, measurement of Carr index confirms that CaCO₃ 95MD is expected to be the better flow additive than DiCafos A150.

Summing up, Example 1 shows superiority of CaCO₃ 95MD over DiCafos A150.

### Example 2 (Flowability of Mixtures)

Two mixtures comprising metformin HCl, a spray dried formulation of vitamin B12 and one of the two flow additives of example 1 (i.e. either DiCafos A150 or CaCO₃ 95MD) were prepared. The weight ratio between metformin HCl and the flow additive was in both cases approximately 4:1.

Flowability of the two mixtures was then tested as described in Example 1.

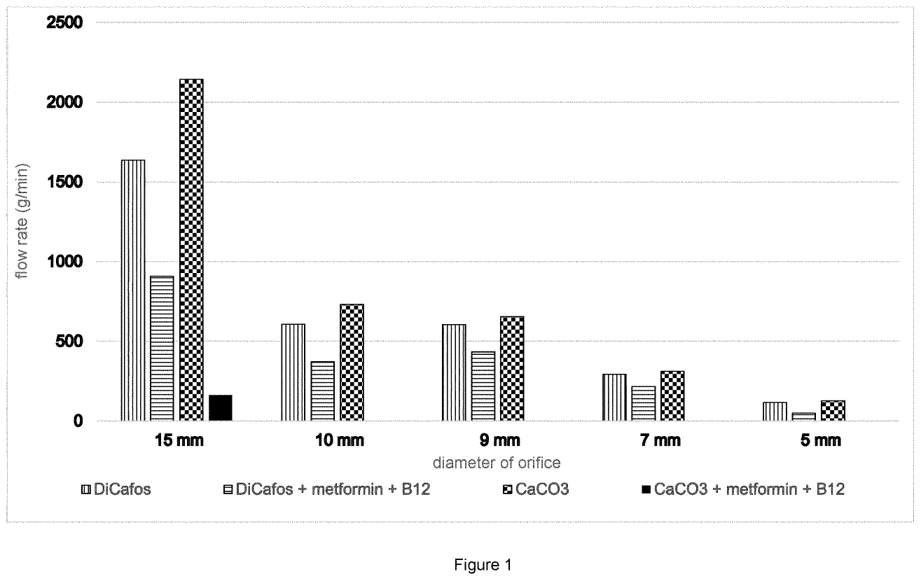

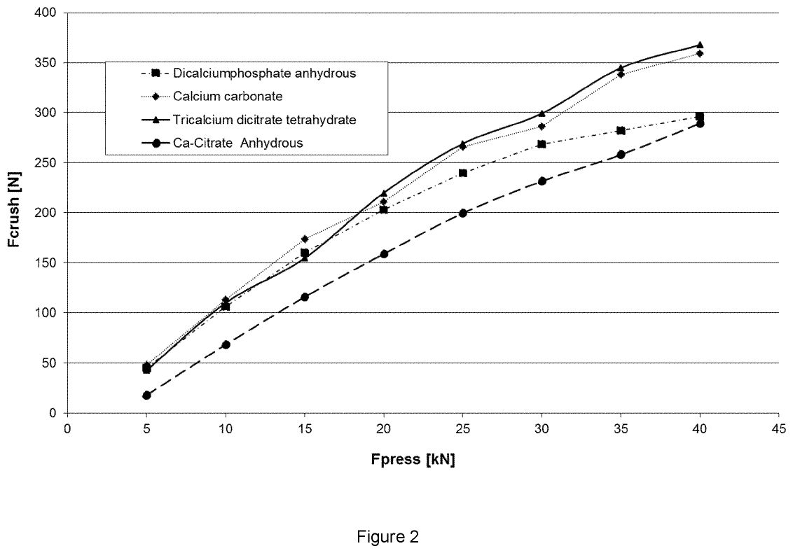

Independent of the size of the orifice, the mixture comprising DiCafos A150 as flow additive had a higher flow rate than the mixture comprising CaCO₃ 95MD (see FIG. 1). This is very surprising as the pretests of Example 1 suggested the opposite.

A summary of the measurements of Example 2 is given in below TABLE 1.

TABLE 1

| | | | | | Mixture comprising | | | | Mixture comprising | |
|---|---|---|---|---|---|---|---|---|---|---|
| | | | | | metformin HCl, | | | | metformin HCl, | |
| | | | | | a spray dried | | | | a spray dried | |
| | | | | | formulation of | | | | formulation of | |
| | | | | | vitamin B12 | | | | vitamin B12 | |
| | | | DiCafos | | and DiCafos | | CaCO3 | | and CaCO3 | |
| | | | A150 | | A150 | | 95MD | | 95MD | |
| Flow rate, | 15 | mm | 1636 | g/min | 909 | g/min | 2143 | g/min | 163 | g/min |
| measured with | 10 | mm | 608 | g/min | 374 | g/min | 732 | g/min | 0 | g/min |
| orifice having | 9 | mm | 606 | g/min | 434 | g/min | 655 | g/min | 0 | g/min |
| the indicated | 7 | mm | 295 | g/min | 217 | g/min | 312 | g/min | 0 | g/min |
| inner diameter | 5 | mm | 117 | g/min | 50 | g/min | 126 | g/min | 0 | g/min |

| | | | | |
|---|---|---|---|---|
| Carr index | 17.4 | 22.9 | 15.8 | 33.7 |

Example 2 does not show that the results of Example 1 are wrong. However, what Example 2 shows is that in the specific case of a blend metformin HCl/vitamin B12, DiCafos A150 increases flowability of more than CaCO₃ 95MD. This is surprising.

DiCafos A150 is a powder comprising aggregates of primary particles.

### Example 3

Four similar tablet mixtures were prepared, each comprising granulated metformin DC 92.6%, a spray dried formulation of vitamin B12 (available at DSM® Nutritional Products), Aerosil 200, magnesium stearate as a lubricant, Prosolv° SMCC90 (available at JRS Pharma) as a binder, and a calcium salt as a flow additive. Thereby, four different kinds of calcium salts were tested: calcium carbonate (95MD available at Particle Dynamics), dicalciumphosphat anhydrous (DiCafos A150, anhydrous, available at Budenheim), tricalcium dicitrate tetrahydrate (available at Merck) and anhydrous calcium citrate (available at Gadot).

For compressing the tablets, a single punch press (Korsch XP-1, available at Korsch, Berlin) was used.

All four tablet mixtures could be successfully compressed into a tablet, regardless which calcium salt had been used. Each of the tablet comprised the same amount of metformin, vitamin B12 (spray dried formulation), Ca²⁺ (100 mg/tablet), Aerosil 200 and lubricant. The amount of binder (Prosolv° SMCC90) per tablet was then chosen such that each of the obtained tablet had a mass of 1500 mg.

Tablet hardness was then measured using a Krämer UTS4 1 apparatus. The obtained compression profiles (Fpress vs. Fcrush) are shown in FIG. 2. Fcrush is the force needed to break a tablet axially. Fpress is the force developed by upper punch during tableting.

### Example 4

Depending on the medical indication, either an extended release (XR) dosage form or an immediate release (IR) dosage form of metformin is prescribed. An example of a commercially available immediate release formulation is Glucophage® IR.

To achieve immediate release, disintegration time of a tablet should be reasonably short.

In example 4, the physical characteristics of four tablets comprising metformin HCl, a spray dried formulation of vitamin B12 and a calcium salt were measured. The result is shown in below TABLE 2:

TABLE 2

| | Tablet 1 | Tablet 2 | Tablet 3 | Tablet 5 |
|---|---|---|---|---|
| Calcium salt | dicalciumphosphate | calcium | tricalcium dicitrate | anhydrous |
| | anhydrous | carbonate | tetrahydrate | calcium citrate |
| Tablet thickness (mm) | 7.61 | 7.60 | 7.90 | 8.34 |
| Tablet weight (mg) | 1507 mg | 1502 mg | 1499 mg | 1508 mg |
| Hardness (N) | 252N | 274N | 275N | 194N |
| Friability (%) | 0.07 | 0.08 | 0.07 | 0.23 |
| Disintegration time (min:s) | 06:06 | 07:00 | 03:07 | 01:07 |

Tablet hardness was measured as described in USP <1217> and EP<2.9.8.> with a Krämer UTS4 1 tester (Kraemer Elektronik GmbH, Darmstadt, Germany). The inventors measured the force needed to break a tablet axially. Presented are the average values of 10 measurements.

Tablet disintegration was characterized according USP<701, 2040> by using a DISI-1 disintegration tester (Charles Ischi PG Pharma Prüftechnik, Zuchwill, Switzerland) in 900 mL demineralized water at 37° C. Six parallel measurements were carried out. Upper limit of disintegration time is 30 min for uncoated tablets (USP <2040>).

Friability, that is closely related to tablet hardness, refers to the extent of weight loss during mechanical abrasion. A maximum loss of no more than 1% of the initial tablet weight is considered acceptable (USP <1216>, EP<2.9.7.>). The inventors tested 10 tablets in an AE-1 Friabilator (Charles Ischi AG Pharma Prüftechnik, Zuchwill, Switzerland) at a rotation speed of 25 rpm for 4 minutes. The weight loss of the tablets was recorded.

Example 4 shows that disintegration time is shorter when anhydrous dicalciumphosphate is used instead of calcium carbonate. A disintegration time of 6 minutes is acceptable and, if necessary, could be shortened by adding a disintegration agent.

### Example 5

In example 5, three different kinds of tablets were prepared. Each tablet comprised 549.9 mg calcium phosphate (anhydrous, available at Emcompress®) and 0.0078 mg vitamin B12. Apart from the source of vitamin B12, the different kinds of tablets were identical.

To investigate the impact on content uniformity, the following three different kinds of vitamin B12 were tested:
Vitamin B12 cryst. (crystalline vitamin B12, available at DSM® Nutritional Products)
Vitamin B12 1% SD (spray dried formulation of vitamin B12, available at DSM® Nutritional Products)
Vitamin B12 0.1% WS (spray dried formulation of vitamin B12, available at DSM® Nutritional Products)

Tablets were compressed with a Korsch XL 100 rotary tableting machine (Korsch A G, Berlin, Germany) using an oblong punch of 22×9 mm and compression force of 20 kN.

Vitamin B12 content uniformity was then evaluated via the standard deviation RSD (%) calculated from 10 individual assay determinations (HPLC analysis conducted by Eurofins®, Germany).

As shown in below TABLE 3, the relative standard deviations (RSD) values relating to the two spray dried formulations of vitamin B12 were below 5%, indicating acceptable content uniformity and hence homogeneous distribution of Vitamin B12 in the tablets. In contrast, content uniformity relating to vitamin B12 crystalline was extremely poor.

TABLE 3

| | | tablet 1 | tablet 2 | tablet 3 |
|---|---|---|---|---|
| weight of tablet (mg) | | 1560.8 | 1553.9 | 1538.3 |
| calcium phosphate anhydrous | | 549.9 | 549.9 | 549.9 |
| (mg/tablet) | | | | |
| source of vitamin B12 | | vitamin B12 | vitamin B12 | vitamin B12 |
| | | cryst. | 1% SD | 0.1% WS |
| amount of source of vitamin | | 0.0078 | 0.78 | 7.8 |
| B12 (mg/tablet) | | | | |
| amount of vitamin B12 per | | 0.0078 | 0.0078 | 0.0078 |
| tablet (mg/tablet) | | | | |
| vitamin B12 content | RSD | 90.8 | 3.3 | 2.0 |
| uniformity | (%) | | | |
| | rating | extremely | good | good |
| | | poor | | |

1. Composition comprising:
a) metformin HCl
b) at least one spray dried formulation of vitamin B12, and
c) at least one flow additive,
wherein said at least one flow additive comprises a calcium salt.

2. Composition according to claim 1, wherein said flow additive is a powder, and wherein said powder preferably comprises aggregates of primary particles, wherein said primary particles comprise or consist of a calcium salt.

3. Composition according to claim 1, wherein less than 10 wt.-% of the flow additive is retained by mesh 40 and more than 30 wt.-% of the flow additive is retained by mesh 100, based on the total weight of the flow additive.

4. Composition according to claim 1, wherein the weight ratio between metformin HCl and the at least one flow additive is from 200:1 to 1:1 and is more preferably from 100:1 to 1:1 and is even more preferably from 50:1 to 1:1 and is most preferably from 30:1 to 1:1.

5. Composition according to claim 1, wherein said calcium salt is calcium phosphate or calcium carbonate, and wherein said calcium salt is preferably calcium phosphate.

6. Composition according to claim 1, wherein said calcium salt is anhydrous calcium phosphate or hydrous calcium phosphate, and wherein said calcium salt is preferably anhydrous calcium phosphate, and wherein said anhydrous calcium phosphate is preferably anhydrous monocalcium phosphate (Ca(H₂PO₄)₂), anhydrous dicalcium phosphate (CaHPO₄) or anhydrous tricalcium phosphate (Ca₃(PO₄)₂), and wherein said calcium salt is most preferably anhydrous dicalcium phosphate (CaHPO₄).

7. Composition according to claim 1, wherein the spray dried formulation of vitamin B12 is obtainable by spray drying of an aqueous solution that comprises vitamin B12 and at least one auxiliary compound, and
wherein said at least one auxiliary compound is preferably selected from the group consisting of sodium citrate, trisodium citrate, citric acid, maltodextrin citric acid and modified food starch.

8. Composition according to claim 1,
wherein the composition is a mixture and wherein said mixture is preferably a powderous mixture.

9. Composition according to claim 8,
wherein said mixture has a Carr index of preferably less than 30, more preferably of less than 28 and most preferably of less than 25, and/or
wherein said mixture has a flow rate of preferably at least 250 g/min, more preferably at least 500 g/min and most preferably at least 1000 g/min when measuring the flow rate of the mixture through a funnel with an orifice that has an inner diameter of 15 mm.

10. Solid pharmaceutical dosage form comprising the composition according to claim 1.

11. Solid pharmaceutical dosage form according to claim 1, wherein said solid pharmaceutical dosage form is an oral solid pharmaceutical dosage form, and
wherein said oral solid pharmaceutical dosage form is preferably a tablet or a capsule, and wherein said tablet is preferably a compressed tablet.

12. Method of manufacturing a fixed-dose combination of metformin HCl and vitamin B12, said method comprising the step of letting the composition according to claim 1 flow down a guiding tool.

13. Method according to claim 12, wherein the composition flows down a tube, hose, a duct, a channel or a conduit.

14. Use of aggregates of primary particles for increasing flowability of a composition comprising metformin HCl and vitamin B12, wherein said primary particles comprise or consist of calcium salt.

15. Use according to claim 14, wherein less than 10 wt.-%, preferably less than 6 wt.-% of the aggregates pass mesh 325, based on the total weight of the aggregates, and/or less than 30 wt.-%, preferably less than 26 wt.-% of the aggregates pass mesh 200, based on the total weight of aggregates, and/or more than 30 wt.-%, preferably more than 35 wt.-% of the aggregates are retained by mesh 100, based on the total weight of the aggregates, and/or wherein less than 10 wt.-%, preferably less than 5 wt.-% of the aggregates are retained by mesh 40, based on the total weight of the aggregates.
